(19) United States
(12) Patent Application Publication
(10) Pub. No.: US 2026/0125376 A1
(43) Pub. Date: May 7, 2026

(54) IRREVERSIBLE INACTIVATORS TARGETING ARAF/MEK COMPLEXES

(71) Applicants: Thomas GERO, Boston, MA (US); Dana-Farber Cancer Institute, Inc., Boston, MA (US)

(72) Inventors: Thomas Gero, Boston, MA (US); David A. Scott, Boston, MA (US); Michael Eck, Boston, MA (US); Tyler Beyett, Boston, MA (US); Javier Vinals-Camallonga, Boston, MA (US)

(21) Appl. No.: 19/117,845

(22) PCT Filed: Oct. 2, 2023
(86) PCT No.: PCT/US2023/075740

§ 371 Date: Apr. 2, 2025

Related U.S. Application Data
(60) Provisional application No. 63/378,160, filed on Oct. 3, 2022.

(51) Int. Cl. C07D 471/04; A61K 31/4184; A61K 31/506; A61K 31/519; C07C 237/34; C07D 235/08; C07D 405/12; C07D 405/14

(52) U.S. Cl. CPC C07D 471/04; A61K 31/4184; A61K 31/506; A61K 31/519; C07C 237/34; C07D 235/08; C07D 405/12; C07D 405/14

(57) ABSTRACT

The disclosure relates to compounds that act as irreversible inactivators that target ARAF/MEK complexes; pharmaceutical compositions comprising the compounds; and methods of treating or preventing kinase-mediated disorders, including cancer and other proliferation diseases.

### RELATED APPLICATIONS

This application claims priority to U.S. Provisional Application No. 63/378,160 filed Oct. 3, 2022, the entire content of which is incorporate by reference.

### STATEMENT REGARDING FEDERALLY SPONSORED RESEARCH OR DEVELOPMENT

This invention was made with government support under Grant No. R35CA242461 awarded by the National Institutes of Health (NIH) and Grant No. F32CA247198 awarded by the National Institutes of Health (NIH). The government has certain rights in the invention.

### BACKGROUND

Mitogen-activated protein kinase kinase (MEK) is the central kinase in the three-tiered RAF/MEK/ERK cascade (also called the MAP kinase cascade). MEK can be activated by one or more of three RAF proteins ARAF, BRAF, or CRAF. Current MEK kinase inhibitors are relatively toxic and poorly tolerated, at least in part because they block most or all signaling through MEK, irrespective of how it was activated. Each of the three RAF isoforms can be mutationally activated in cancer. BRAF is very frequently mutated in cancer, whereas ARAF and CRAF are much less frequently mutated. As such, it would be desirable to have MEK inhibitors that could block signaling in a RAF-selective manner; i.e., MEK inhibitors that preferentially inhibit BRAF/MEK or ARAF/MEK, with relative sparing of signaling through the other isoforms.

Treatment of cancers or other diseases by preferential inhibition of ARAF/MEK signaling is preferred. Activating ARAF mutations, though rare, have been identified in diverse cancers (doi: 10.1016/j.cell.2018.03.035). Additionally, activating ARAF mutations occur in Langerhans Cell Histiocytosis (doi: 10.1182/blood-2013-06-511139) and central conducting lymphatic anomaly (doi: 10.1038/s41591-019-0479-2). Activating mutations in ARAF have also been identified in intrahepatic cholangiocarcinoma (DOI: 10.1038/ncomms7087) and altered expression of ARAF has been implicated in development of hepatocellular carcinoma (Shilo et al. RNA 20:505-515). Treatment of these or other indications with the ARAF/MEK inhibitors disclosed herein could selectively block the signaling through ARAF. Additionally, signaling through ARAF has been identified as a mechanism of resistance to the Type II RAF inhibitor belvarafenib (https://doi.org/10.1038/s41586-021-03515-1). Treatment with an irreversible ARAF/MEK inhibitor of the present disclosure could be used to treat belvarafenib-resistant cancers in which this mechanism is at play. Finally, these compounds could be useful as a tool for studying the role of ARAF is cell signaling, development, or other contexts.

The compounds provided herein are designed to irreversibly inactivate ARAF signaling through the ARAF/MEK pathway. They bind in a pocket in the ARAF/MEK complex that is formed primarily by MEK but is closed on one end by ARAF. The ARAF portion of the pocket includes a cysteine residue, ARAFCys514, that is unique to ARAF. These inactivators are designed to form a covalent bond with Cys514, resulting in irreversible inactivation of ARAF. They are not expected to irreversibly inactivate BRAF or CRAF, because these isoforms lack the target cysteine residue.

Therefore, the compounds disclosed herein provide the potential for more selective inhibition of signaling through ARAF/MEK complexes, which should be more effective and/or better tolerated than current agents that indiscriminately inhibit MEK signaling.

### SUMMARY

In an aspect, provided herein is a compound of Formula I:

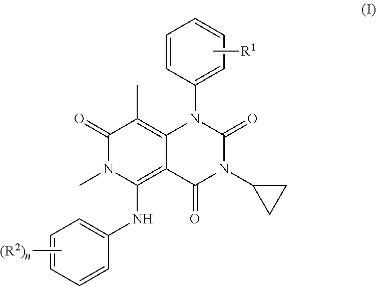

or a pharmaceutically acceptable salt thereof; wherein the variables are defined herein.

In an embodiment, the compound of Formula I is a compound of Formula Ia:

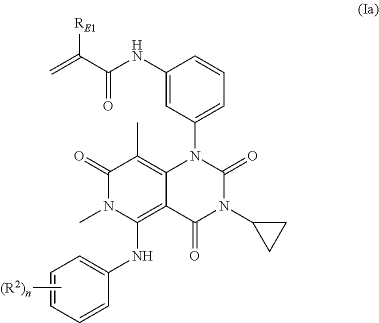

or a pharmaceutically acceptable salt thereof.

In another aspect, provided herein is a compound of Formula II:

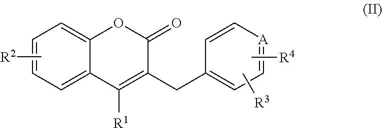

or a pharmaceutically acceptable salt thereof; wherein the variables are defined herein.

In an embodiment, the compound of Formula II is a compound of Formula Ila:

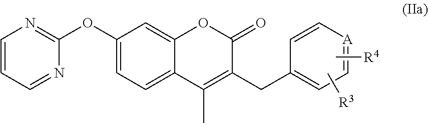

or a pharmaceutically acceptable salt thereof.

In an aspect, provided herein is a method of treating cancer or a proliferation disease, comprising administering to a subject in need thereof an effective amount of a compound disclosed herein or a pharmaceutical composition comprising a compound disclosed herein and a pharmaceutically acceptable carrier. In one embodiment, the cancer is melanoma or carcinoma.

In another aspect, provided herein is a method of inactivating the activity of ARAF in a subject in need thereof, comprising administering to the subject an effective amount of a compound disclosed herein or a pharmaceutical composition comprising a compound disclosed herein and a pharmaceutically acceptable carrier.

The disclosure also provides a kit comprising a compound capable of inactivating ARAF activity selected from a compound of the present disclosure, or a pharmaceutically acceptable salt thereof, and instructions for use in treating cancer.

### DETAILED DESCRIPTION

The present disclosure describes the selective inhibition of aberrant signaling through the RAF/MEK/ERK pathway caused by inappropriate and/or undesirable activation of ARAF, and more effective, durable, potent inhibition of ARAF/MEK complexes as compared with inhibition by current MEK inhibitors.

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or +10%, including ±5%, 1%, and +0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with ARAF/MEK complexes an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “inactivating” and other forms thereof (e.g., “inactivate,” “inactivates,” etc.) refers to preventing or partially preventing downstream signaling of a target protein or the target protein in complex with other proteins; preventing or partially preventing further activation of other proteins; and/or preventing or partially preventing further binding of other proteins through said protein or complex.

As used herein, the term “inactivator” refers to an agent that inactivates a target protein or protein complex as defined above.

As used herein, the term “compound” refers to is a chemical substance composed of many identical molecules wherein the atoms of the molecules are linked together by covalent bonds.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “MEK” refers to mitogen-activated protein kinase kinase (alternately referred to as MAP2K or MAPKK), which is a kinase enzyme that phosphorylates mitogen-activated protein kinase (MAPK).

As used herein, the term “RAF” refers to a family of three serine/threonine-specific protein kinases that are related to retroviral oncogenes. The three RAF protein kinases are A-RAF, B-RAF, and C-RAF.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein, the term “haloalkyl” refers to an alkyl group, as defined above, substituted with one or more halo substituents, wherein alkyl and halo are as defined herein. Haloalkyl includes, by way of example, chloromethyl, trifluoromethyl, bromoethyl, chlorofluoroethyl, and the like.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “haloalkenyl” refers to an alkenyl moiety as defined above, wherein the alkenyl moiety is further substituted with a halo group as defined below.

As used herein, the term “alkynyl” refers to a monovalent group derived from a hydrocarbon moiety containing, in certain embodiments, from two to six, or two to eight carbon atoms having at least one carbon-carbon triple bond. The alkynyl group may or may not be the point of attachment to another group. The term “alkynyl” includes, but is not limited to, ethynyl, 1-propynyl, 1-butynyl, heptynyl, octynyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, cycloalkyl is C₃₋₁₀ cycloalkyl. In another embodiment, cycloalkyl is C₃₋₆ cycloalkyl.

As used herein, the term “cycloalkenyl” means a non-aromatic carbocyclic system that is partially saturated having 1, 2 or 3 rings wherein such rings may be fused, and wherein at least one ring contains an sp² carbon-carbon bond. The term “cycloalkenyl” includes, but is not limited to, cyclopropenyl, cyclobutenyl, cyclopentenyl, cyclohexenyl, bicyclo[3.1.0]hexenyl, spiro[3.3]heptanenyl, and bicyclo[1.1.1]pentenyl. In an embodiment, cycloalkenyl is C₃₋₁₀ cycloalkyl. In another embodiment, cycloalkenyl is C₃₋₆ cycloalkenyl.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocyclyl also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydro-furanyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo[2.1.1]-hexanyl, 6-azabicyclo[3.1.1]heptanyl, 2-azabicyclo[2.2.1]heptanyl, 3-azabicyclo[3.1.1]heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro-[3.5]nonanyl, 3-oxaspiro[5.3]nonanyl, and 8-oxabicyclo[3.2.1]octanyl. In an embodiment, heterocycloalkyl is 3-10 membered heterocycloalkyl. In another embodiment, heterocycloalkyl is 3-6 membered heterocycloalkyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta-[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, heteroaryl is 5-10 membered heteroaryl. In another embodiment, heteroaryl is 5-6 membered heteroaryl.

It is to be understood that if an aryl, heteroaryl, cycloalkyl, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the term “substituted” means that an atom or group of atoms has replaced hydrogen as the substituent attached to another group.

### Compounds

Provided herein are compounds that are irreversible inhibitors of MEK/ARAF complexes useful in the treatment of kinase-mediated disorders, including cancer and other proliferation diseases.

In an aspect, provided herein is a compound of Formula I:

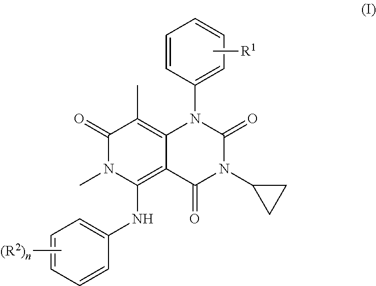

or a pharmaceutically acceptable salt thereof;
   wherein:
each R² is independently selected from the group consisting of C₁₋₆ alkyl, C₁₋₆ haloalkyl, halo, CN, OH, NH₂, NH(C₁₋₆ alkyl), and N(C₁₋₆ alkyl)₂;
n is 1 or 2;
R¹ is selected from the group consisting of

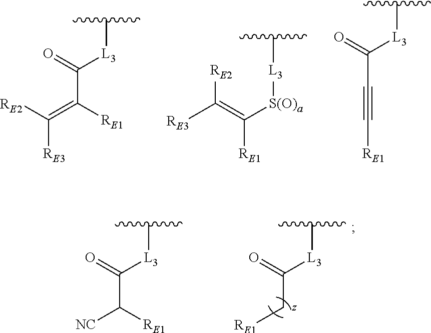

L₃ is selected from the group consisting of a bond, C₁-C₄ alkyl, and NH;
each of R_{E1}, R_{E2}, and R_{E3} is independently selected from the group consisting of H, halo, CN, and C₁-C₆ alkyl;
a is 1 or 2; and
each z is independently 0, 1, or 2.

In an embodiment, R¹ is

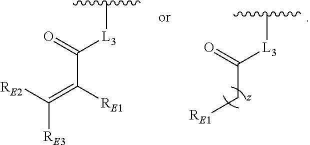

In another embodiment, the compound of Formula I is a compound of Formula Ia:

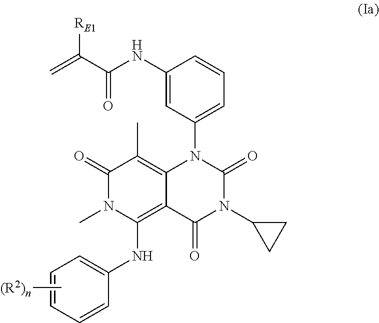

or a pharmaceutically acceptable salt thereof;
wherein R_{E1} is H or halo.

In yet another embodiment, n is 1. In still another embodiment, n is 2.

In an embodiment, R² is halo.

In another embodiment, R¹ is selected from the group consisting of:

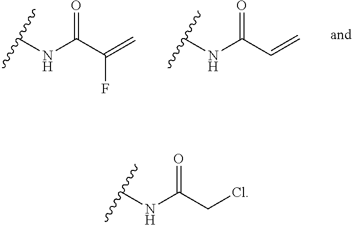

In another embodiment, the compound of Formula I is selected from the group consisting of the compounds of Table 1 below.

TABLE 1

| Compound No | Structure |
|---|---|
| 011 | 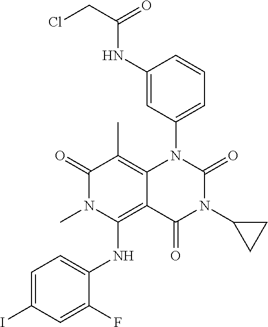 [SMILES: Cc1c(=O)n(C)c(Nc2ccc(I)cc2F)c2c(=O)n(C3CC3)c(=O)n(-c3cccc(NC(=O)CCl)c3)c12] |
| 012 | 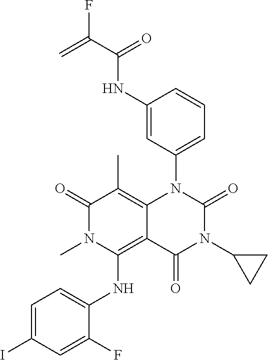 [SMILES: C=C(F)C(=O)Nc1cccc(-n2c(=O)n(C3CC3)c(=O)c3c(Nc4ccc(I)cc4F)n(C)c(=O)c(C)c32)c1] |
| 022 | 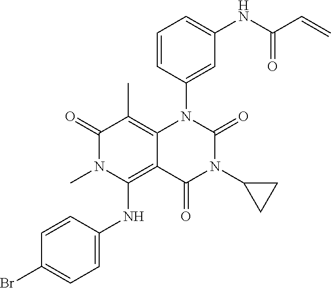 [SMILES: C=CC(=O)Nc1cccc(-n2c(=O)n(C3CC3)c(=O)c3c(Nc4ccc(Br)cc4)n(C)c(=O)c(C)c32)c1] |
| 023 | 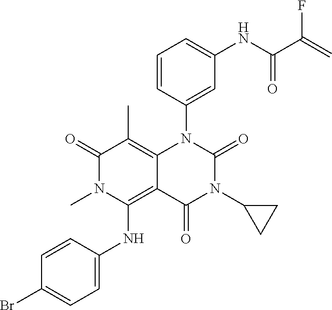 [SMILES: C=C(F)C(=O)Nc1cccc(-n2c(=O)n(C3CC3)c(=O)c3c(Nc4ccc(Br)cc4)n(C)c(=O)c(C)c32)c1] |
| 024 | 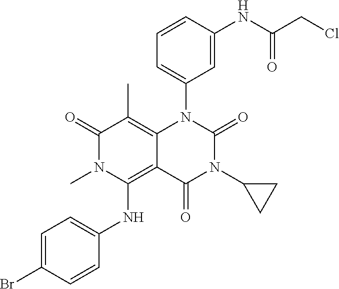 [SMILES: Cc1c(=O)n(C)c(Nc2ccc(Br)cc2)c2c(=O)n(C3CC3)c(=O)n(-c3cccc(NC(=O)CCl)c3)c12] |

or a pharmaceutically acceptable salt thereof.

In another embodiment, the compound of Formula I is

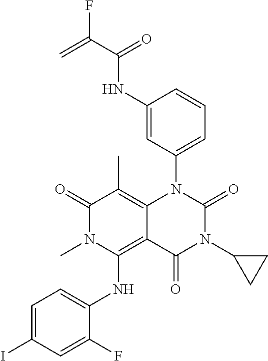 [SMILES: C=C(F)C(=O)Nc1cccc(-n2c(=O)n(C3CC3)c(=O)c3c(Nc4ccc(I)cc4F)n(C)c(=O)c(C)c32)c1]

or a pharmaceutically acceptable salt thereof.

In another aspect, provided herein is a compound selected from the group consisting of the compounds of Table 2 below.

TABLE 2

| Compound No | Structure |
|---|---|
| 005 | 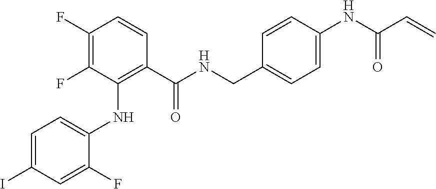 [SMILES: C=CC(=O)Nc1ccc(CNC(=O)c2ccc(F)c(F)c2Nc2ccc(I)cc2F)cc1] |
| 006 | 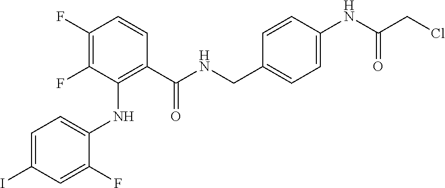 [SMILES: O=C(CCl)Nc1ccc(CNC(=O)c2ccc(F)c(F)c2Nc2ccc(I)cc2F)cc1] |

or a pharmaceutically acceptable salt thereof.

In yet another aspect, provided herein is a compound selected from the group consisting of the compounds of Table 3 below.

TABLE 3

| Compound No | Structure |
|---|---|
| 007 | 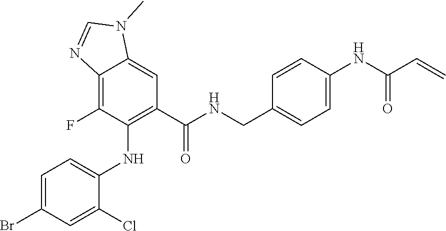 [SMILES: C=CC(=O)Nc1ccc(CNC(=O)c2cc3c(ncn3C)c(F)c2Nc2ccc(Br)cc2Cl)cc1] |
| 008 | 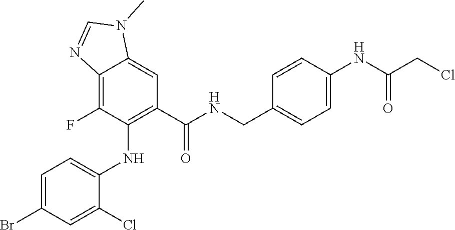 [SMILES: Cn1cnc2c(F)c(Nc3ccc(Br)cc3Cl)c(C(=O)NCc3ccc(NC(=O)CCl)cc3)cc21] |
| 009 | 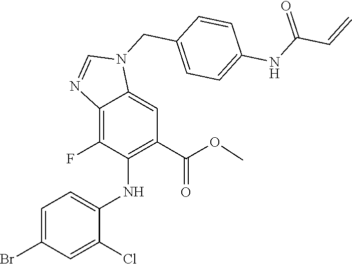 [SMILES: C=CC(=O)Nc1ccc(Cn2cnc3c(F)c(Nc4ccc(Br)cc4Cl)c(C(=O)OC)cc32)cc1] |
| 010 | 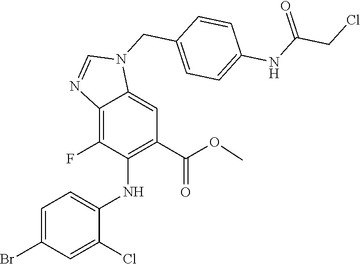 [SMILES: COC(=O)c1cc2c(ncn2Cc2ccc(NC(=O)CCl)cc2)c(F)c1Nc1ccc(Br)cc1Cl] |

or a pharmaceutically acceptable salt thereof.

In still another aspect, provided herein is a compound of Formula II:

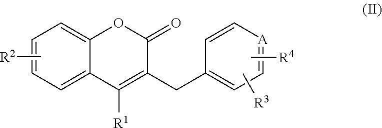

or a pharmaceutically acceptable salt thereof;
   wherein
A is N, CR³, or CR⁴;
R¹ is H or C₁₋₆ alkyl;
R² is selected from the group consisting of H, halo, 6-10 membered aryl, 5-10 membered heteroaryl, 3-10 membered cycloalkyl, 3-10 membered cycloalkenyl, 4-10 membered heterocycloalkyl, OR⁶, NR⁶R⁶, SO₂R⁶, SO₂NHR⁶, NHSO₂R⁶, C(O)OR⁶, C(O)NHR⁶, C(O)R⁶, C₁-C₆ alkyl, C₂-C₆ alkenyl, and C₂-C₆ alkynyl, wherein aryl, heteroaryl, heterocycloalkyl, cycloalkyl, alkyl, alkenyl, and alkynyl are optionally substituted one, two, or three times with R⁷;
each R³ is independently selected from the group consisting of H, halo, C₁₋₃ alkyl, C. 3 haloalkyl, and C₁₋₃ alkoxy; and
each R⁶ is independently selected from the group consisting of H, C₁₋₆ alkyl, C₁₋₆ alkenyl, C₁₋₆ alkynyl, C₁₋₆ haloalkyl, 6-10 membered aryl, 5-6 membered heteroaryl, 3-6 membered cycloalkyl, and 3-6 membered heterocycloalkyl;
each R⁷ is independently selected from the group consisting of halo, OH, NH₂, NH(C₁₋₆ alkyl), N(C₁₋₆ alkyl)₂, and CN;
R⁴ is selected from the group consisting of

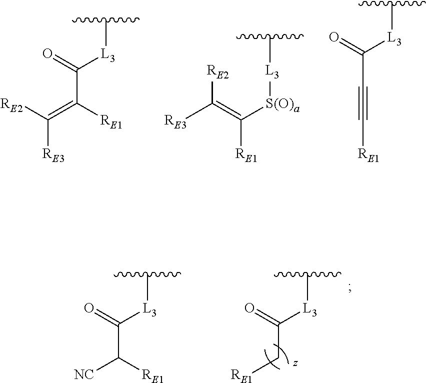

L₃ is selected from the group consisting of a bond, C₁-C₄ alkyl, and NH;
each of R_{E1}, R_{E2}, and R_{E3} is independently selected from the group consisting of H, halo, CN, and C₁-C₆ alkyl;
a is 1 or 2; and
each z is independently 0, 1, or 2.

In an embodiment, the compound of Formula II is a compound of Formula Ila:

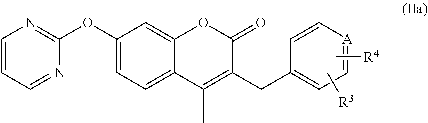

or a pharmaceutically acceptable salt thereof.

In another embodiment, R³ is H or halo.

In yet another embodiment, A is N. In still another embodiment, A is CR³. In another embodiment, A is CR⁴.

In an embodiment, R⁴ is

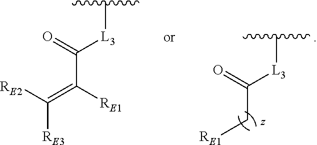

In another embodiment, R⁴ is selected from the group consisting of:

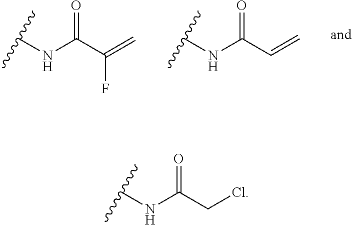

In an embodiment, the compound of Formula II is selected from the group consisting of the compounds of Table 4 below.

TABLE 4

| Compound No | Structure |
|---|---|
| 001 | 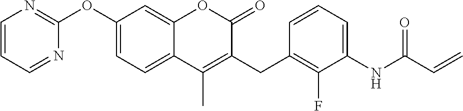 [SMILES: C=CC(=O)Nc1cccc(Cc2c(C)c3ccc(Oc4ncccn4)cc3oc2=O)c1F] |
| 002 | 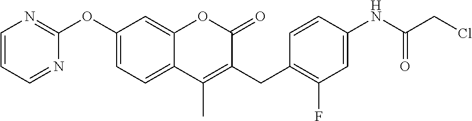 [SMILES: Cc1c(Cc2ccc(NC(=O)CCl)cc2F)c(=O)oc2cc(Oc3ncccn3)ccc12] |
| 003 | 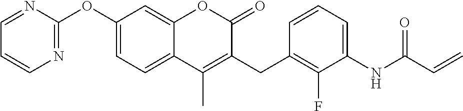 [SMILES: C=CC(=O)Nc1cccc(Cc2c(C)c3ccc(Oc4ncccn4)cc3oc2=O)c1F] |
| 004 | 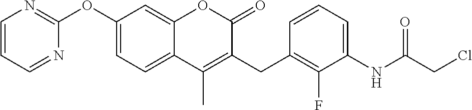 [SMILES: Cc1c(Cc2cccc(NC(=O)CCl)c2F)c(=O)oc2cc(Oc3ncccn3)ccc12] |
| 013 | 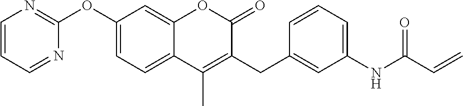 [SMILES: C=CC(=O)Nc1cccc(Cc2c(C)c3ccc(Oc4ncccn4)cc3oc2=O)c1] |
| 014 | 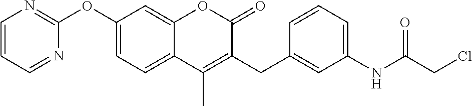 [SMILES: Cc1c(Cc2cccc(NC(=O)CCl)c2)c(=O)oc2cc(Oc3ncccn3)ccc12] |
| 015 | 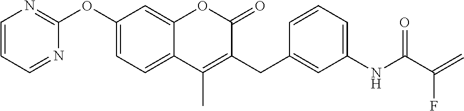 [SMILES: C=C(F)C(=O)Nc1cccc(Cc2c(C)c3ccc(Oc4ncccn4)cc3oc2=O)c1] |
| 016 | 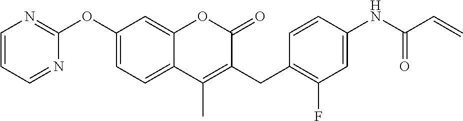 [SMILES: C=CC(=O)Nc1ccc(Cc2c(C)c3ccc(Oc4ncccn4)cc3oc2=O)c(F)c1] |
| 017 | 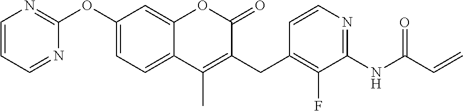 [SMILES: C=CC(=O)Nc1nccc(Cc2c(C)c3ccc(Oc4ncccn4)cc3oc2=O)c1F] |
| 018 | 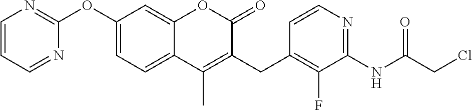 [SMILES: Cc1c(Cc2ccnc(NC(=O)CCl)c2F)c(=O)oc2cc(Oc3ncccn3)ccc12] |
| 019 | 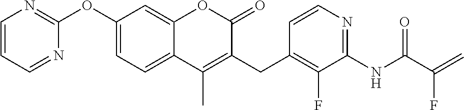 [SMILES: C=C(F)C(=O)Nc1nccc(Cc2c(C)c3ccc(Oc4ncccn4)cc3oc2=O)c1F] |
| 020 | 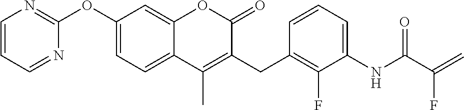 |
| 021 | 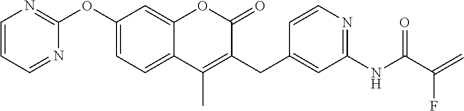 [SMILES: C=C(F)C(=O)Nc1cc(Cc2c(C)c3ccc(Oc4ncccn4)cc3oc2=O)ccn1] |

or a pharmaceutically acceptable salt thereof.

In an aspect, provided herein is a pharmaceutical composition comprising any one of the compounds disclosed herein, or a pharmaceutically acceptable salt thereof, and at least one pharmaceutically acceptable carrier.

The compounds disclosed herein may exist as tautomers and optical isomers (e.g., enantiomers, diastereomers, diastereomeric mixtures, racemic mixtures, and the like).

It is generally well known in the art that any compound that will be converted in vivo to provide a compound disclosed herein is a prodrug within the scope of the present disclosure.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

### Methods of Treatment

In an aspect, provided herein is a method of treating cancer in subject in need thereof, comprising administering to the subject a therapeutically effective amount of a compound disclosed herein.

In an embodiment, the subject is resistant to treatment with a RAF inhibitor. In another embodiment, the subject has been previously treated with a RAF inhibitor. In yet another embodiment, the RAF inhibitor is a RAF dimer inhibitor. In still another embodiment, the RAF dimer inhibitor is belvarafenib.

In an embodiment, the cancer is melanoma.

In another embodiment, the cancer is carcinoma. In yet another embodiment, the carcinoma is selected from the group consisting of uterine corpus endometrial carcinoma, cholangiocarcinoma, and esophageal adenocarcinoma. In an embodiment, the carcinoma is uterine corpus endometrial carcinoma. In another embodiment, the carcinoma is cholangiocarcinoma. In yet another embodiment, the carcinoma is esophageal adenocarcinoma.

In an embodiment, the cancer is selected from the group consisting of lung cancer, colon cancer, breast cancer, endometrial cancer, thyroid cancer, glioma, squamous cell carcinoma, and prostate cancer. In another embodiment, the cancer is non-small cell lung cancer (NSCLC).

In another embodiment, the compound selectively inhibits the ARAF/MEK complex in the subject. In yet another embodiment, the compound irreversibly inhibits ARAF in the subject. In still another embodiment, the compound irreversibly inactivates ARAF in the subject.

In another aspect, provided herein is a method of inactivating ARAF in a subject in need thereof comprising administering to the subject a therapeutically effective amount of a compound provided herein.

In an embodiment, the compound binds in a pocket of an ARAF/MEK complex. In another embodiment, the ARAF portion of the pocket includes a cysteine residue that is ARAF^{Cys514}. In yet another embodiment, ARAF is characterized as having a modification on Cys514. In still another embodiment, the compound covalently binds to Cys514.

In an embodiment, the compound selectively inhibits the ARAF/MEK complex. In another embodiment, the subject is diagnosed with a cancer. In yet another embodiment, the cancer is carcinoma. In still an embodiment, the carcinoma is selected from the group consisting of uterine corpus endometrial carcinoma, cholangiocarcinoma, and esophageal adenocarcinoma.

In an embodiment of the methods, the subject is diagnosed with Langerhans Cell Histiocytosis or a central conducting lymphatic anomaly. In another embodiment of the methods, the compound irreversibly inactivates ARAF.

In another aspect, provided herein is a method of inhibiting a kinase in a subject in need thereof, comprising administering to the subject a therapeutically effective amount of a compound provided herein.

In yet another aspect, provided herein is a method of treating or preventing a kinase-mediated disorder in a subject in need thereof, comprising administering to the subject a therapeutically effective amount of a compound of the present disclosure.

In an embodiment, the kinase is MEK. In another embodiment, the kinase is RAF. In yet another embodiment, the kinase is ARAF. In still another embodiment, the kinase is an ARAF/MEK complex.

Potency of the inhibitor can be determined by EC₅₀ value. A compound with a lower EC₅₀ value, as determined under substantially similar conditions, is a more potent inhibitor relative to a compound with a higher EC₅₀ value.

Potency of the inhibitor can also be determined by IC₅₀ value. A compound with a lower IC₅₀ value, as determined under substantially similar conditions, is a more potent inhibitor relative to a compound with a higher IC₅₀ value.

In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the cancer is lung cancer, breast cancer, glioma, squamous cell carcinoma, or prostate cancer. In still further embodiments, the cancer is non-small cell lung cancer.

In certain embodiments, the cancer is resistant to a RAF inhibitor. In an embodiment, the RAF inhibitor is a RAF dimer inhibitor. In another embodiment, the RAF dimer inhibitor is belvarafenib.

In other embodiments, the subject is administered an additional therapeutic agent. In other embodiments, the compound and the additional therapeutic agent are administered simultaneously or sequentially.

In an embodiment of the methods disclosed herein, the subject is a human.

In another aspect, the disclosure provides a compound disclosed herein, or a pharmaceutically acceptable salt thereof, for use in the manufacture of a medicament for treating or preventing a disease in which ARAF and/or MEK plays a role.

In an aspect, provided herein is a method of treating or preventing a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia. The term “cancer” includes, but is not limited to, the following cancers: myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the disclosure, the present disclosure provides for the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

The term “cancerous cell” as provided herein, includes a cell afflicted by any one of the above-identified conditions.

The disclosure further provides a method for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, adrenoleukodystrophy (ALD), Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliferative disease, or a neurodegenerative disease, comprising administering an effective amount of a compound, or a pharmaceutically acceptable composition comprising a compound, to a subject in need thereof.

The activity of the compounds and compositions of the present disclosure may be assayed in vitro, in vivo, or in a cell line. Detailed conditions for assaying a compound utilized in this disclosure as an inhibitor of various kinases are set forth in the Examples below.

For any of the above uses, the required dosage will vary depending on the mode of administration, the particular condition to be treated and the effect desired.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting kinase activity selected from one or more compounds of disclosed herein, or pharmaceutically acceptable salts thereof, and instructions for use in treating cancer.

In another aspect, the disclosure provides a kit comprising a compound capable of inactivating ARAF activity selected from a compound disclosed herein, or a pharmaceutically acceptable salt thereof.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1: Synthesis Procedures

### Abbreviations

ACN acetonitrile
AIBN azobisisobutyronitrile
DCM dichloromethane
DMF dimethylformamide
DMSO dimethylsulfoxide
EtOAc ethyl acetate
HATU 1-[bis(dimethylamino)methylene]-1H-1,2,3-triazolo[4,5-b]pyridinium 3-oxide hexafluorophosphate
NBS N-bromosuccinimide
RT room temperature
TFA trifluoroacetic acid
THE tetrahydrofuran

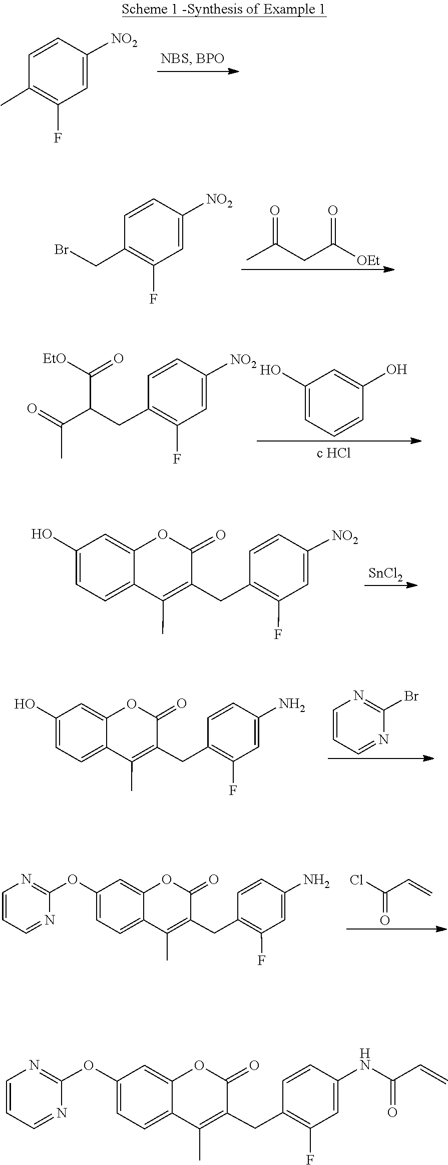

### Example 1

### 1-(Bromomethyl)-2-fluoro-4-nitrobenzene

A solution of 2-fluoro-1-methyl-4-nitrobenzene (2.4 g, 15 mmol), N-bromosuccinimide (3.2 g, 18 mmol), and AIBN (246 m g, 1.5 mmol) in 50 mL of CCl₄ was heated to reflux for 16 hours. After cooling, the mixture was filtered. The filtrate was concentrated under reduced pressure and the residue purified by flash chromatography, 0-20% EtOAc/hexane to give the title compound (2.07 g, 59%). ¹H NMR (500 MHz, CDCl₃-d) 5 ppm 8.06 (dd, 1H) 7.98 (dd, 1H) 7.63 (t, 1H) 4.55 (s, 2H).

### Ethyl 2-(2-fluoro-4-nitrobenzyl)-3-oxobutanoate

A suspension of NaH (326 mg, 65% in oil, 8.8 mmol) in anhydrous THE (20 mL) under N₂ was cooled to 0° C. in an ice bath. Ethylacetoacetate (1.2 mL, 8.8 mmol) was added and the mixture was stirred at RT for 30 min. A solution of 1-(bromomethyl)-2-fluoro-4-nitrobenzene (2.07 g, 8.8 mmol) in THE (15 mL) was added and the reaction was stirred for 16 hours at RT. The reaction mixture was treated with 1.5 N HCl (50 mL) and extracted with EtOAc (2×75 mL). The combined organic extracts were washed with water and brine, dried over Na₂SO₄, filtered and concentrated under reduced pressure. The residue was purified by flash chromatography, 0-30% EtOAc/hexane to give the title compound (1.54 g, 61%). ¹H NMR (500 MHz, DMSO-d₆) δ ppm 8.09 (dd, 1H) 8.04 (dd, 1H) 7.62 (t, 1H) 7.15-7.25 (m, 1H) 4.07 (m, 3H) 3.17 (m, 2H) 2.23 (s, 3H) 1.11 (t, 3H).

### 3-(2-Fluoro-4-nitrobenzyl)-7-hydroxy-4-methyl-2H-chromen-2-one

Ethyl 2-(2-fluoro-4-nitrobenzyl)-3-oxobutanoate (1.5 g, 4.55 mmol) in sulfuric acid (3 mL) at 0° C. was treated with resorcinol (501 mg, 4.55 mmol). The reaction mixture was stirred for 72 hours at RT and quenched with ice water (30 mL). The resulting precipitate was filtered, washed with water and dried to give the title compound, (1.18 g, 79%). ¹H NMR (500 MHz, DMSO-d₆) δ ppm 10.51 (s, 1H) 8.09 (dd, 1H) 7.97 (dd, 1H) 7.70 (d, 1H) 7.44 (t, 1H) 6.83 (dd, 1H) 6.73 (d, 1H) 4.04 (s, 2H) 2.42 (s, 3H).

### 3-(4-Amino-2-fluorobenzyl)-7-hydroxy-4-methyl-2H-chromen-2-one

A mixture of 3-(2-fluoro-4-nitrobenzyl)-7-hydroxy-4-methyl-2H-chromen-2-one (1.17 g, 3.55 mmol), tin (II) chloride dihydrate (4.01 g, 17.76 mmol), and EtOAc (50 mL) was heated to reflux for 7 hours. After cooling, the reaction mixture was quenched with saturated sodium bicarbonate (30 mL) and extracted with ethyl acetate (2×50 mL). The combined organic extracts were washed with brine, dried over Na₂SO₄, filtered and concentrated under reduced pressure to give the title compound (480 mg, 45%), used without further purification.

### 3-(4-Amino-2-fluorobenzyl)-4-methyl-7-(pyrimidin-2-yloxy)-2H-chromen-2-one (Aniline 1)

A mixture of 3-(4-amino-2-fluorobenzyl)-7-hydroxy-4-methyl-2H-chromen-2-one (480 mg, 1.6 mmol), 2-chloropyrimidine (728 mg, 604 mmol), Cs₂CO₃ (627 mg, 1.9 mmol) and DMF (15 mL), was heated to 100° C. for 40 min. After cooling, the reaction mixture was poured into water (150 mL) and extracted with of EtOAc (3×100 mL). The combined organic extracts were washed with saturated brine, dried over Na₂SO₄, filtered and concentrated under reduced pressure. The residue was purified by flash chromatography eluting with 0-100% EtOAc/hexane to give the title compound (182 mg, 30%). ¹H NMR (500 MHz, DMSO-d₆) δ ppm 8.69 (d, 2H) 7.88 (d, 1H) 7.36 (d, 1H) 7.34 (m, 1H) 7.25 (dd, 1H) 6.78 (m, 1H) 6.29 (m, 2H) 5.22 (s, 2H) 3.80 (s, 2H) 2.45 (s, 3H); MS m/z: 378.34 [M+1]⁺.

### Example 1 N-(3-Fluoro-4-((4-methyl-2-oxo-7-(pyrimidin-2-yloxy)-2H-chromen-3-yl)methyl)phenyl) acrylamide

A solution of 3-(4-amino-2-fluorobenzyl)-4-methyl-7-(pyrimidin-2-yloxy)-2H-chromen-2-one (50 mg, 0.13 mmol), diisopropylethylamine (34.5 uL, 0.20 mmol), and DCM (2 mL) at 0° C. was treated with acryloyl chloride (12.8 uL, 0.16 mmol). The reaction mixture was stirred for 15 minutes and concentrated under reduced pressure. The residue was purified by reverse phase-HPLC, 0-80% ACN/H₂O (TFA modifier) to give the title compound (42 mg, 74%). ¹H NMR (500 MHz, DMSO-d₆) δ: 10.29 (s, 1H) 8.68 (d, 2H) 7.91 (d, 1H) 7.70 (dd, 1H) 7.38 (d, 1H) 7.34 (t, 1H) 7.27 (dd, 1H) 7.20 (dd, 1H) 7.12 (t, 1H) 6.40 (dd, 1H) 6.27 (dd, 1H) 5.77 (dd, 1H) 3.95 (s, 2H) 2.48 (s, 3H); MS m/z: 432.32 [M+1]⁺.

TABLE 4
Examples 2-10
Examples 2-10 were prepared in a similar manner to Example 1 from the corresponding
aniline and acryloyl chloride or chloroacetyl chloride.

| | | | NMR | |
|---|---|---|---|---|
| Cmpd | | | 500 MHz, | |
| No. | | LCMS | DMSO-d₆ | Aniline |
| 2 | 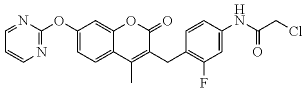 [SMILES: Cc1c(Cc2ccc(NC(=O)CCl)cc2F)c(=O)oc2cc(Oc3ncccn3)ccc12] 2-Chloro-N-(3-fluoro-4-((4- | 454.29 | 10.45 (s, 1H) 8.68 (d, 2H) 7.91 (d, 1H) 7.59 (dd, 1H) 7.38 (d, 1H) 7.34 (t, 1H) 7.27 (dd, 1H) 7.18 (dd, 1H) 7.13 (t, 1H) 4.25 (s, 2H) 3.95 (s, 2H) 2.48 | Aniline 1 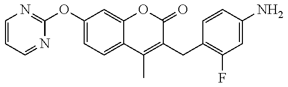 [SMILES: Cc1c(Cc2ccc(N)cc2F)c(=O)oc2cc(Oc3ncccn3)ccc12] |
| | methyl-2-oxo-7-(pyrimidin-2- | | (s, 3H) | 3-(4-Amino-2- |
| | yloxy)-2H-chromen-3- | | | fluorobenzyl)-4-methyl- |
| | yl)methyl)phenyl)acetamide | | | 7-(pyrimidin-2-yloxy)-2H- |
| | | | | chromen-2-one |
| 3 | 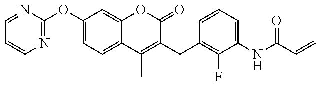 [SMILES: C=CC(=O)Nc1cccc(Cc2c(C)c3ccc(Oc4ncccn4)cc3oc2=O)c1F] N-(2-Fluoro-3-((4-methyl-2-oxo- 7-(pyrimidin-2-yloxy)-2H- | 432.11 | 9.92 (s, 1H) 8.69 (d, 2H) 7.92 (d, 1H) 7.87 (m, 1H) 7.37 (d, 1H) 7.34 (t, 1H) 7.27 (dd, 1H) 7.06 (t, 1H) 6.96 (m, 1H) 6.62 (dd, 1H) 6.28 (dd, 1H) 5.78 (dd, | Aniline 2 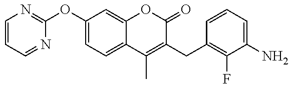 [SMILES: Cc1c(Cc2cccc(N)c2F)c(=O)oc2cc(Oc3ncccn3)ccc12] |
| | chromen-3- | | 1H) 4.03 (s, 2H) 3 | 3-(3-Amino-2- |
| | yl)methyl)phenyl)acrylamide | | protons masked by | fluorobenzyl)-4-methyl- |
| | | | solvent peak | 7-(pyrimidin-2-yloxy)-2H- |
| | | | | chromen-2-one |
| 4 | 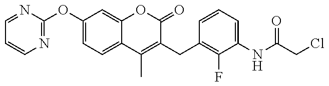 [SMILES: Cc1c(Cc2cccc(NC(=O)CCl)c2F)c(=O)oc2cc(Oc3ncccn3)ccc12] 2-Chloro-N-(2-fluoro-3-((4- methyl-2-oxo-7-(pyrimidin-2- | 454.04 | 10.07 (s, 1H) 8.69 (d, 2H) 7.92 (d, 1H) 7.75 (m, 1H) 7.37 (d, 1H) 7.34 (t, 1H) 7.27 (dd, 1H) 7.07 (t, 1H) 6.98 (m, 1H) 4.35 (s, 2H) 4.03 (s, 2H) 2.49 (s, 3H) | Aniline 2 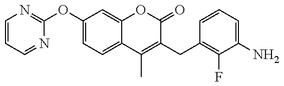 [SMILES: Cc1c(Cc2cccc(N)c2F)c(=O)oc2cc(Oc3ncccn3)ccc12] |
| | yloxy)-2H-chromen-3- | | | 3-(3-Amino-2- |
| | yl)methyl)phenyl)acetamide | | | fluorobenzyl)-4-methyl- |
| | | | | 7-(pyrimidin-2-yloxy)-2H- |
| | | | | chromen-2-one |
| 5 | 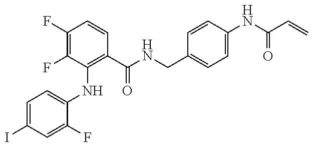 [SMILES: C=CC(=O)Nc1ccc(CNC(=O)c2ccc(F)c(F)c2Nc2ccc(I)cc2F)cc1] N-(4-Acrylamidobenzyl)-3,4- difluoro-2-((2-fluoro-4- | 552.33 | 10.11 (s, 1H) 9.27 (s, 1H) 9.23 (m, 1H) 7.61 (m, 4H) 7.39 (dd, 1H) 7.25 (d, 2H) 7.22 (m, 1H) 6.72 (m, 1H) 6.42 (dd, 1H) 6.25 (dd, 1H) 5.74 (dd, 1H) 4.39 (d, 2H) | Aniline 3 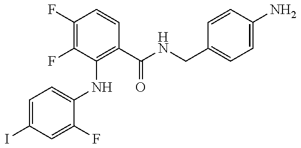 [SMILES: Nc1ccc(CNC(=O)c2ccc(F)c(F)c2Nc2ccc(I)cc2F)cc1] |
| | iodophenyl)amino)benzamide | | | N-(4-Aminobenzyl)-3,4- |
| | | | | difluoro-2-((2-fluoro-4- |
| | | | | iodophenyl)amino)benza- |
| | | | | mide |
| 6 | 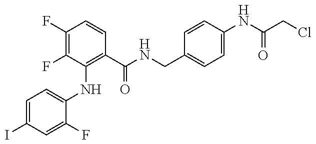 [SMILES: O=C(CCl)Nc1ccc(CNC(=O)c2ccc(F)c(F)c2Nc2ccc(I)cc2F)cc1] N-(4-(2-Chloroacetamido) | 573.95 | 10.27 (s, 1H) 9.26 (s, 1H) 9.23 (m, 1H) 7.61 (m, 2H) 7.52 (d, 2H) 7.38 (dd, 1H) 7.26 (d, 2H) 7.23 (m, 1H) 6.70 (m, 71H) 4.39 (8d, 2H) 4.24 (s, 2H) | Aniline 3 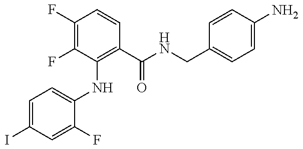 [SMILES: Nc1ccc(CNC(=O)c2ccc(F)c(F)c2Nc2ccc(I)cc2F)cc1] |
| | benzyl)-3,4-difluoro-2-((2- | | | N-(4-Aminobenzyl)-3,4- |
| | fluoro-4- | | | difluoro-2-((2-fluoro-4- |
| | iodophenyl)amino)benzamide | | | iodophenyl)amino)benza- |
| | | | | mide |
| 7 | 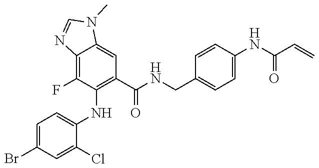 [SMILES: C=CC(=O)Nc1ccc(CNC(=O)c2cc3c(ncn3C)c(F)c2Nc2ccc(Br)cc2Cl)cc1] N-(4-Acrylamidobenzyl)-5-((4- bromo-2-chlorophenyl)amino)- 4-fluoro-1-methyl-1H- | 558.34 | 10.11 (s, 1H) 9.10 (m, 1H) 8.41 (s, 2H) 7.87 (s, 1H) 7.61 (d, 1H) 7.58 (d, 2H) 7.28 (dd, 1H) 7.24 (d, 2H) 6.43 (dd, 1H) 6.42 (m, 1H) 6.25 (dd, 1H) 5.75 (dd, 1H) 4.42 (d, 2H) 3.92 (s, 3H) | Aniline 4 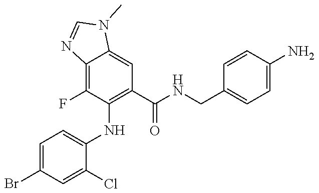 [SMILES: Cn1cnc2c(F)c(Nc3ccc(Br)cc3Cl)c(C(=O)NCc3ccc(N)cc3)cc21] |
| | benzo[d]imidazole-6- | | | N-(4-Aminobenzyl)-5- |
| | carboxamide | | | ((4-bromo-2- |
| | | | | chlorophenyl)amino)-4- |
| | | | | fluoro-1-methyl-1H- |
| | | | | benzo[d]imidazole-6- |
| | | | | carboxamide |
| 8 | 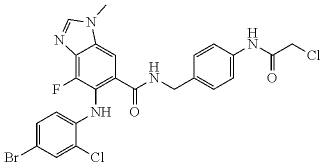 [SMILES: Cn1cnc2c(F)c(Nc3ccc(Br)cc3Cl)c(C(=O)NCc3ccc(NC(=O)CCl)cc3)cc21] 5-((4-Bromo-2- chlorophenyl)amino)-N-(4-(2- chloroacetamido)benzyl)-4- | 580.31 | 10.26 (s, 1H) 9.10 (m, 1H) 8.40 (s, 1H) 8.38 (s, 1H) 7.86 (s, 1H) 7.61 (d, 1H) 7.50 (d, 2H) 7.28 (dd, 1H) 7.24 (d, 2H) 6.40 (dd, 1H) 4.42 (d, 2H) 4.24 (s, 2H) 3.91 (s, 3H) | Aniline 4 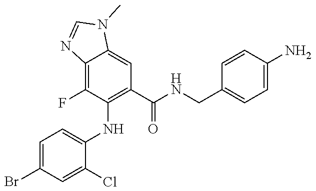 [SMILES: Cn1cnc2c(F)c(Nc3ccc(Br)cc3Cl)c(C(=O)NCc3ccc(N)cc3)cc21] |
| | fluoro-1-methyl-1H- | | | N-(4-Aminobenzyl)-5- |
| | benzo[d]imidazole-6- | | | ((4-bromo-2- |
| | carboxamide | | | chlorophenyl)amino)-4- |
| | | | | fluoro-1-methyl-1H- |
| | | | | benzo[d]imidazole-6- |
| | | | | carboxamide |
| 9 | 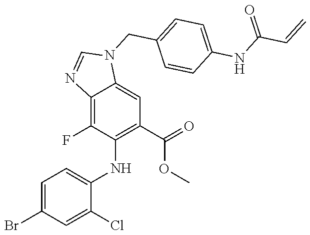 [SMILES: C=CC(=O)Nc1ccc(Cn2cnc3c(F)c(Nc4ccc(Br)cc4Cl)c(C(=O)OC)cc32)cc1] Methyl 1-(4-acrylamidobenzyl)- | 559.39 | 10.19 (s, 1H) 8.67 (s, 1H) 8.09 (s, 1H) 8.04 (s, 1H) 7.66 (d, 2H) 7.63 (d, 1H) 7.32 (d, 2H) 7.26 (dd, 1H) 6.45 (dd, 1H) 6.41 (dd, 1H) 6.25 (dd, 1H) 5.75 (dd, 1H) 5.58 (s, 2H) 3.80 (s, 3H) | Aniline 5 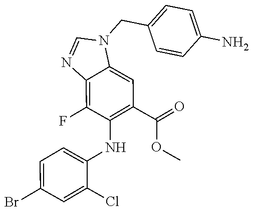 [SMILES: COC(=O)c1cc2c(ncn2Cc2ccc(N)cc2)c(F)c1Nc1ccc(Br)cc1Cl] |
| | 5-((4-bromo-2- | | | Methyl 1-(4- |
| | chlorophenyl)amino)-4-fluoro- | | | aminobenzyl)-5-((4- |
| | 1H-benzo[d]imidazole-6- | | | bromo-2- |
| | carboxylate | | | chlorophenyl)amino)-4- |
| | | | | fluoro-1H- |
| | | | | benzo[d]imidazole-6- |
| | | | | carboxylate |
| 10 | 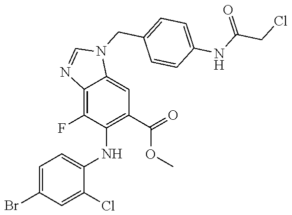 [SMILES: COC(=O)c1cc2c(ncn2Cc2ccc(NC(=O)CCl)cc2)c(F)c1Nc1ccc(Br)cc1Cl] Methyl 5-((4-bromo-2- | 581.31 | 10.34 (s, 1H) 8.67 (s, 1H) 8.09 (s, 1H) 8.03 (s, 1H) 7.63 (d, 1H) 7.58 (d, 2H) 7.32 (d, 2H) 7.27 (dd, 1H) 6.45 (dd, 1H) 5.59 (s, 2H) 4.23 (s, 2H) 3.80 (s, 3H) | Aniline 5 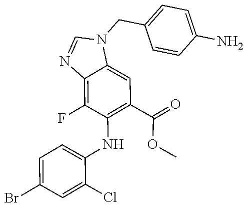 [SMILES: COC(=O)c1cc2c(ncn2Cc2ccc(N)cc2)c(F)c1Nc1ccc(Br)cc1Cl] |
| | chlorophenyl)amino)-1-(4-(2- | | | Methyl 1-(4- |
| | chloroacetamido)benzyl)-4- | | | aminobenzyl)-5-((4- |
| | fluoro-1H-benzo[d]imidazole-6- | | | bromo-2- |
| | carboxylate | | | chlorophenyl)amino)-4- |
| | | | | fluoro-1H- |
| | | | | benzo[d]imidazole-6- |
| | | | | carboxylate |

Aniline 2. 3-(3-Amino-2-fluorobenzyl)-4-methyl-7-(pyrimidin-2-yloxy)-2H-chromen-2-one was prepared according to the procedures in Hyohdoh et al, ACS Med. Chem. Lett. 2013, 4, 1059-1063.
Aniline 3. N-(4-Aminobenzyl)-3,4-difluoro-2-((2-fluoro-4-iodophenyl)amino)benzamide tert-Butyl (4-((3,4-difluoro-2-((2-fluoro-4-iodophenyl)amino)benzamido)methyl)phenyl)carbamate

A mixture of 3,4-difluoro-2-((2-fluoro-4-iodophenyl)amino)benzoic acid (500 mg, 1.27 mmol), tert-butyl (4-(aminomethyl)phenyl)carbamate (489 mg, 2.20 mmol), diisopropylethylamine (333 uL, 1.9 mmol), HATU (967 mg, 2.54 mmol), and DMF (4 mL) was stirred at RT for 1 hour. The reaction mixture was poured into water (50 mL) and extracted with of EtOAc (2×50 mL). The combined organic extracts were washed with saturated brine, dried over Na₂SO₄, filtered and conc under reduced pressure. The crude material was purified by flash chromatography, eluting with 0-50% EtOAc/hexane to give the title compound (558 mg, 73.5%). ¹H NMR (500 MHz, DMSO-d₆) δ ppm 9.27 (m, 2H) 9.19 (m, 1H) 7.59 (m, 2H) 7.38 (m, 3H) 7.17 (m, 3H) 6.70 (m, 1H) 4.34 (d, 2H) 1.47 (s, 9H); MS m/z: 598.37 [M+1]⁺.

### N-(4-Aminobenzyl)-3,4-difluoro-2-((2-fluoro-4-iodophenyl)amino)benzamide

A solution of tert-butyl (4-((3,4-difluoro-2-((2-fluoro-4-iodophenyl)amino)benzamido)methyl)phenyl) carbamate (360 mg, 0.6 mmol) in DCM (1 mL) and TFA (1 mL) was stirred at RT for 5 minutes. Solvents were removed under reduced pressure to give a quantitative yield of the title compound. ¹H NMR (500 MHz, DMSO-d₆) δ ppm 9.26 (m, 2H) 7.61 (m, 2H) 7.39 (dd, 1H) 7.28 (d, 2H) 7.23 (dd, 1H) 7.08 (d, 2H) 6.71 (m, 1H) 4.39 (d, 2H); MS m/z: 498.23 [M+1]⁺.
Aniline 4. N-(4-Aminobenzyl)-5-((4-bromo-2-chlorophenyl)amino)-4-fluoro-1-methyl-1H-benzo[d]imidazole-6-carboxamide
Aniline 4 was prepared in a similar manner to Aniline 3 from 5-((4-bromo-2-chlorophenyl)amino)-4-fluoro-1-methyl-1H-benzo[d]imidazole-6-carboxylic acid and tert-butyl (4-(aminomethyl)phenyl)carbamate. ¹H NMR (500 MHz, DMSO-d₆) δ ppm 9.18 (m, 1H) 8.44 (s, 1H) 8.35 (s, 1H) 7.88 (s, 1H) 7.62 (d, 1H) 7.34 (d, 2H) 7.28 (dd, 1H) 7.18 (d, 2H) 6.42 (dd, 1H) 4.46 (d, 2H) 3.92 (s, 3H); MS m/z: 502.26 [M+1]+.
Aniline 5. Methyl 1-(4-aminobenzyl)-5-((4-bromo-2-chlorophenyl)amino)-4-fluoro-1H-benzo[d]imidazole-6-carboxylate Methyl 5-((4-bromo-2-chlorophenyl)amino)-1-(4-((tert-butoxycarbonyl)amino)benzyl)-4-fluoro-1H-benzo[d]imidazole-6-carboxylate

A mixture of methyl 5-((4-bromo-2-chlorophenyl)amino)-4-fluoro-1H-benzo[d]imidazole-6-carboxylate (272 mg, 0.68 mmol), tert-butyl (4-(bromomethyl)phenyl)carbamate (196 mg, 0.68 mmol), Cs₂CO₃ (223 mg, 0.68 mmol) in DMF (3 mL) was stirred for 3 hour at 35° C. Additional tert-butyl (4-(bromomethyl)phenyl)carbamate (98 mg, 0.34 mmol) and Cs₂CO₃ (112 mg, 0.34 mmol) were added and the reaction was stirred for 16 hours at 35° C. The entire reaction mixture was purified by reverse phase HPLC, 0-80% ACN/H₂O (TFA modifier) to give the title compound, (80 mg, 20%). ¹H NMR (500 MHz, DMSO-d₆) δ ppm 9.38 (s, 1H) 8.65 (s, 1H) 8.09 (s, 1H) 8.02 (s, 1H) 7.63 (d, 1H) 7.43 (d, 2H) 7.27 (m, 3H) 6.44 (dd, 1H) 5.53 (s, 2H) 3.80 (s, 3H) 1.46 (s, 9H); MS m/z: 603.41 [M+1]+.

### Methyl 1-(4-aminobenzyl)-5-((4-bromo-2-chlorophenyl)amino)-4-fluoro-1H-benzo[d]imidazole-6-carboxylate

A solution of methyl 5-((4-bromo-2-chlorophenyl)amino)-1-(4-((tert-butoxycarbonyl)amino)benzyl)-4-fluoro-1H-benzo[d]imidazole-6-carboxylate (48 mg, 0.08 mmol) in DCM (1 mL) and TFA (1 mL) was stirred at RT for 20 minutes. Solvents were removed under reduced pressure to give the title product in quantitative yield. ¹H NMR (500 MHz, DMSO-d₆) δ ppm 8.62 (s, 1H) 8.33 (s, 1H) 8.14 (s, 1H) 7.66 (d, 1H) 7.23 (dd, 1H) 7.20 (d, 2H) 7.06 (d, 2H) 6.43 (dd, 1H) 5.54 (s, 2H) 3.83 (s, 3H); MS m/z: 502.68 [M+1]+.

### Example 11 2-Chloro-N-(3-(3-cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)acetamide

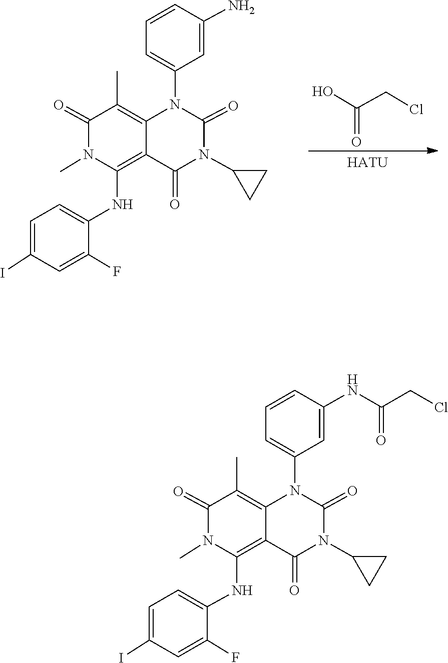

A mixture of 1-(3-aminophenyl)-3-cyclopropyl-5-((2-fluoro-4-iodophenyl)amino)-6,8-dimethylpyrido[4,3-d]pyrimidine-2,4,7(1H,3H,6H)-trione (30 mg, 0.15 mmol), HATU (114 mg, 0.30 mmol), chloroacetic acid (15 mg, 0.165 mmol), diisopropylethylamine (78 uL, 0.45 mmol), and DMF (0.5 mL) was stirred at RT for 30 minutes. The entire reaction mixture was purified by reverse phase HPLC, 0-80% ACN/H₂O (TFA modifier) to give the title compound (14 mg, 15%). ¹H NMR (500 MHz, DMSO-d₆) δ ppm 11.07 (s, 1H) 10.48 (s, 1H) 7.79 (dd, 1H) 7.63 (d, 1H), 7.62 (d, 1H) 7.56 (dd, 1H) 7.42 (t, 1H) 7.11 (dd, 1H) 6.93 (t, 1H) 4.27 (d, 2H) 3.08 (s, 3H) 2.63 (m) 1.26 (s, 3H) 0.96 (m, 2H) 0.68 (m, 2H); MS m/z: 649.69 [M+1]+.
1-(3-Aminophenyl)-3-cyclopropyl-5-((2-fluoro-4-iodophenyl)amino)-6,8-dimethylpyrido[4,3-d]pyrimidine-2,4,7(1H,3H,6H)-trione was prepared according to the procedures in Abe et al, ACS Med. Chem. Lett. 2011, 2, 320-324.

### Example 12 N-(3-(3-Cyclopropyl-5-((2-fluoro-4-iodophenyl)amino)-6,8-dimethyl-2,4,7-trioxo-3,4,6,7-tetrahydropyrido[4,3-d]pyrimidin-1(2H)-yl)phenyl)-2-fluoroacrylamide

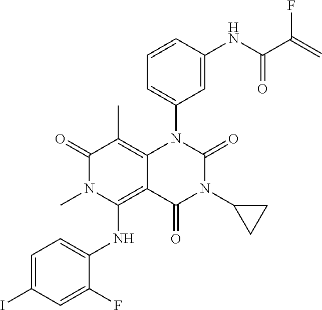 [SMILES: C=C(F)C(=O)Nc1cccc(-n2c(=O)n(C3CC3)c(=O)c3c(Nc4ccc(I)cc4F)n(C)c(=O)c(C)c32)c1]

Example 12 was prepared in a similar manner to Example 11 from 1-(3-aminophenyl)-3-cyclopropyl-5-((2-fluoro-4-iodophenyl)amino)-6,8-dimethylpyrido[4,3-d]pyrimidine-2,4,7(1H,3H,6H)-trione and 2-fluoroacrylic acid. ¹H NMR (500 MHz, DMSO-d₆) δ ppm 11.00-11.16 (m, 1H) 10.39-10.56 (m, 1H) 7.77-7.83 (m, 1H) 7.72-7.77 (m, 2H) 7.54-7.59 (m, 1H) 7.39-7.47 (m, 1H) 7.12-7.20 (m, 1H) 6.89-6.96 (m, 1H) 5.63-5.82 (m, 1H) 5.36-5.52 (m, 1H) 3.05-3.12 (m, 3H) 2.59-2.67 (m, 1H) 1.21-1.30 (m, 3H) 0.90-1.01 (m, 2H) 0.61-0.73 (m, 2H); MS m/z: 646.06 [M+1]*.

TABLE 5
Examples 13-15
Examples 13-15 were prepared in a similar manner to either Example 1 or Example 11
from 3-(3-aminobenzyl)-4-methyl-7-(pyrimidin-2-yloxy)-2H-chromen-2-one (Aniline 6) and
acryloyl chloride, chloroacetyl chloride, or 2-fluoroacrylic acid.

| Cmpd | | | NMR |
|---|---|---|---|
| No. | | LCMS | 500 MHz, DMSO-d₆ |
| 13 | 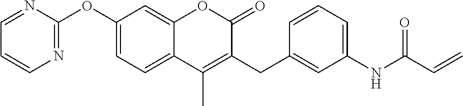 [SMILES: C=CC(=O)Nc1cccc(Cc2c(C)c3ccc(Oc4ncccn4)cc3oc2=O)c1] | 414.30 | 10.07 (br s, 1H) 8.68 (d, 2H) 7.92 (d, 1H) 7.62 (d, 1H) 7.40 (s, 1H) 7.39 (d, 1H) 7.34 (t, 1H) 7.27 (dd, 1H) 7.24 (d, 1H) 7.00 (d, 1H) 6.39 (dd, 1H) 6.23 (dd, 1H) 5.72 (dd, 1H) 3.99 (s, 2H) 3.17 (s, 3H) |
| | N-(3-((4-Methyl-2-oxo-7-(pyrimidin-2- | | |
| | yloxy)-2H-chromen-3- | | |
| | yl)methyl)phenyl)acrylamide | | |
| 14 | 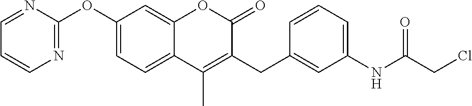 [SMILES: Cc1c(Cc2cccc(NC(=O)CCl)c2)c(=O)oc2cc(Oc3ncccn3)ccc12] | 436.27 | 10.22 (br s, 1H) 8.68 (d, 2H) 7.92 (d, 1H) 7.53 (d, 1H) 7.38 (d, 1H) 7.34 (d, 1H) 7.32 (m, 1H) 7.27 (dd, 1H) 7.25 (d, 1H) 7.02 (d, 1H) 4.20 (s, 2H) 3.99 (s, 2H) 3.17 (s, 3H) |
| | 2-Chloro-N-(3-((4-methyl-2-oxo-7- | | |
| | (pyrimidin-2-yloxy)-2H-chromen-3- | | |
| | yl)methyl)phenyl)acetamide | | |
| 15 | 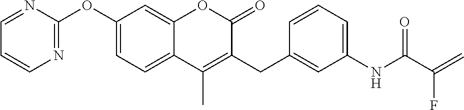 | 432.23 | 10.21 (br s, 1H) 8.68 (d, 2H) 7.92 (d, 1H) 7.63 (d, 1H) 7.50 (s, 1H) 7.39 (d, 1H) 7.33 (t, 1H) 7.28 (dd, 1H) 7.25 (d, 1H) 7.06 (d, 1H) 5.69 (dd, 1H) 5.39 (dd, 1H) 4.00 (s, 2H) Three protons masked by water peak |
| | 2-Fluoro-N-(3-((4-methyl-2-oxo-7- | | |
| | (pyrimidin-2-yloxy)-2H-chromen-3- | | |
| | yl)methyl)phenyl)acrylamide | | |

Aniline 6. 3-(3-Aminobenzyl)-4-methyl-7-(pyrimidin-2-yloxy)-2H-chromen-2-one

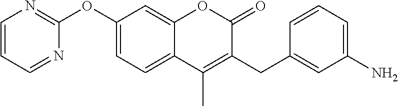 [SMILES: Cc1c(Cc2cccc(N)c2)c(=O)oc2cc(Oc3ncccn3)ccc12]

### 3-(3-aminobenzyl)-4-methyl-7-(pyrimidin-2-yloxy)-2H-chromen-2-one

Aniline 6 was prepared in a similar manner to Aniline 2, from 3-nitrotoluene. ¹H NMR (500 MHz, DMSO-d₆) δ ppm 8.69 (d, 2H) 7.89 (d, 1H) 7.36 (d, 1H) 7.34 (t, 2H) 7.26 (dd, 1H) 6.91 (dd, 1H) 6.38 (m, 3H) 4.96 (br s, 2H) 3.85 (s, 2H) 2.46 (s, 3H).

### Example 2: a Mass Spectrometry-Based Assay for Covalent Labeling of ARAF

Covalent labeling of ARAF by compounds of the invention was measured using a mass spectrometry-based assay. The ARAF/MEK protein complex was incubated at room temperature for 2 hours with desired inhibitors at a concentration ratio of 1:10. Following incubation, proteins were denatured in 8 M urea. Formic acid was added to a final 1% and desalted over C₄ resin prior to LCMS analysis. Denatured proteins (10 μg per sample) were analyzed by LC-MS via a U3000 RSLC coupled to an Orbitrap Eclipse mass spectrometer (Thermo Scientific, Waltham, MA, USA). Proteins were eluted off a ES811A (Thermo Scientific, Waltham, MA, USA) 15-cm C₄ column with a 5-50% gradient of MeCN in 1% FA. MS scans (range=600-2000 m/z) were obtained in the orbitrap at 120,000 resolution with 4 microscans. The Xtract function in Freestyle software (Thermo Scientific, Waltham, MA, USA) was used to deconvolute charge states averaged across the elution window. Fractional labeling of ARAF was measured by quantitating the unlabeled ARAF and ARAF shifted by the mass expected for formation of a covalent adduct of ARAF with the compound of interest. The results of this assay are shown in Table 6.

TABLE 6
Mass Spectrometry-based measurement
of covalent labeling of ARAF

| Example | % labeling |
|---|---|
| 1 | 70 |
| 2 | 50 |
| 3 | 85 |
| 4 | 100 |
| 5 | 10 |
| 6 | 40 |
| 7 | 0 |
| 8 | 40 |
| 9 | 0 |
| 10 | 75 |
| 11 | 100 |
| 12 | 100 |

### Example 3. Inhibition of ARAF Kinase Activity

The ability of compounds of the invention to inhibit phosphorylation of MEK by ARAF was measured in a TR-FRET-based kinase assay using purified ARAF and MEK proteins. An active, chimeric ARAF protein was employed for this study. This ARAF protein included residues 274-606 of human ARAF, including the kinase domain and complete C-terminal region, fused to human 14-3-3 epsilon (ARAF/14-3-3 chimera). Amino acids corresponding to residues 209-302 of human ARAF (with the sequence Ser-Gly-Tyr-Tyr) were mutated to Ser-Ser-Asp-Asp in this ARAF/14-3-3 chimera. The ARAF/14-3-3 chimera protein was found to form active dimers upon expression and purification from Sf9 insect cells using a baculoviral/insect cell expression approach. In this assay, the ability of compounds of interest to inhibit phosphorylation of MEK1 by ARAF was measured by incubating 250 nM unphosphorylated full-length MEK1(D190N) with 75 nM ARAF/14-3-3 chimera and inhibitor of interest at a series of 12 concentrations (from 10 μM to 1.3 nM) for 30 minutes in reaction buffer (50 mM HEPES pH 7.0, 5 mM MgCl₂, 1 mM MnCl₂, 0.01% BSA, 2 mM TCEP, 0.1 mM NaVO₄) at room temperature. Reactions were initiated by addition of 200 μM ATP and allowed to proceed for 1 hour. Reactions were quenched using detection buffer from HTRF-KinEASE kit (Cisbio). Signal was read with a PHERAstar plate reader (excitation=337 nm, emission=620 nm and 665 nm). IC₅₀ values were determined using Graphpad Prism software from the 12 point concentration-response data using a three-parameter dose-response fit with the Hill slope constrained to −1.

TABLE 7

| Compound No. | ARAF IC₅₀ (μM) |
|---|---|
| 012 | 0.21 |
| 011 | 0.49 |
| 004 | 0.06 |
| 003 | 0.13 |

As can be seen from the data in Table 7, above, the compounds provided herein inactivate ARAF, thus preventing ARAF from phosphorylating MEK.

The disclosed subject matter is not to be limited in scope by the specific embodiments and examples described herein. Indeed, various modifications of the disclosure in addition to those described will become apparent to those skilled in the art from the foregoing description and accompanying FIGURES. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1.-41. (canceled)

42. A compound of Formula I: 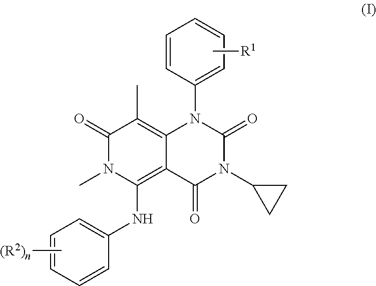
or a pharmaceutically acceptable salt thereof;
wherein
each R² is independently selected from the group consisting of C₁₋₆ alkyl, C₁₋₆ haloalkyl, halo, —CN, —OH, —NH₂, —NH(C₁₋₆ alkyl), and —N(C₁₋₆ alkyl)₂;
n is 1 or 2;
R¹ is selected from the group consisting of 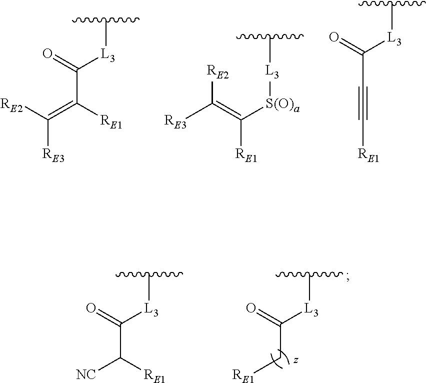
L₃ is selected from the group consisting of a bond, C₁-C₄ alkyl, and NH;
each of R_{E1}, R_{E2}, and R_{E3} is independently selected from the group consisting of H, halo, —CN, and C₁-C₆ alkyl;
a is 1 or 2; and
each z is independently 0, 1, or 2, provided that the compound of Formula I is not

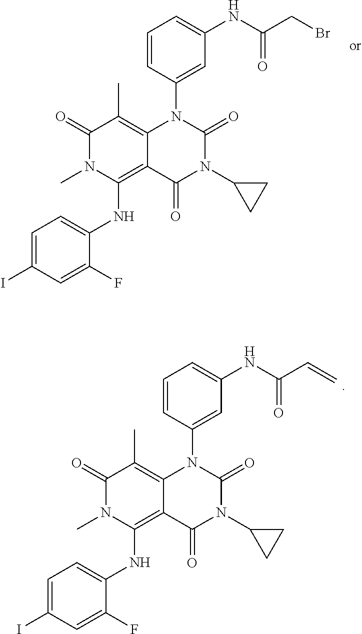 [SMILES: Cc1c(=O)n(C)c(Nc2ccc(I)cc2F)c2c(=O)n(C3CC3)c(=O)n(-c3cccc(NC(=O)CBr)c3)c12 ; C=CC(=O)Nc1cccc(-n2c(=O)n(C3CC3)c(=O)c3c(Nc4ccc(I)cc4F)n(C)c(=O)c(C)c32)c1]

43. The compound of claim 42, wherein the compound of Formula I is a compound of Formula Ia: 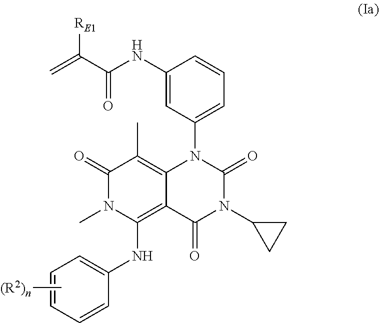
or a pharmaceutically acceptable salt thereof, wherein R_{E1} is H or halo and wherein R² is halo.

44. The compound of claim 42, wherein the compound of Formula I is selected from the group consisting of 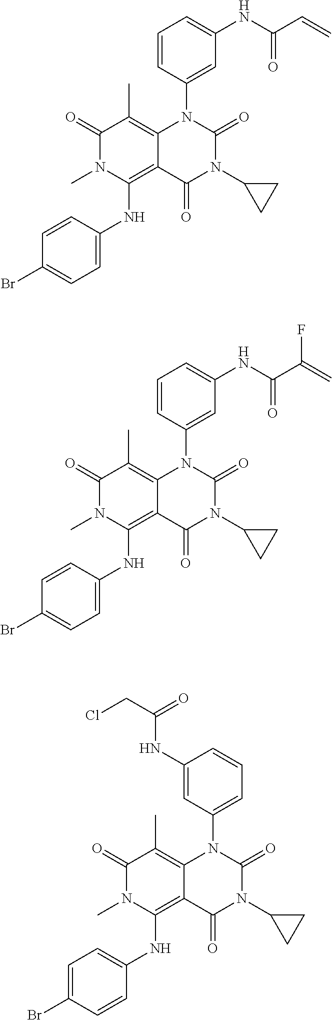 [SMILES: C=CC(=O)Nc1cccc(-n2c(=O)n(C3CC3)c(=O)c3c(Nc4ccc(Br)cc4)n(C)c(=O)c(C)c32)c1 ; C=C(F)C(=O)Nc1cccc(-n2c(=O)n(C3CC3)c(=O)c3c(Nc4ccc(Br)cc4)n(C)c(=O)c(C)c32)c1 ; Cc1c(=O)n(C)c(Nc2ccc(Br)cc2)c2c(=O)n(C3CC3)c(=O)n(-c3cccc(NC(=O)CCl)c3)c12]
or a pharmaceutically acceptable salt thereof.

45. A pharmaceutical composition comprising a compound of claim 42, or a pharmaceutically acceptable salt thereof, and a pharmaceutically acceptable carrier.

46. A method of inactivating ARAF in a subject in need thereof comprising administering to the subject a therapeutically effective amount of a compound of claim 42.

47. A method of treating an ARAF-mediated cancer in a subject in need thereof, comprising administering to the subject a therapeutically effective amount of a compound of claim 42, wherein the ARAF-mediated cancer may be melanoma or a carcinoma, including uterine corpus endometrial carcinoma, cholangiocarcinoma, and esophageal adenocarcinoma.

48. A compound selected from the group consisting of 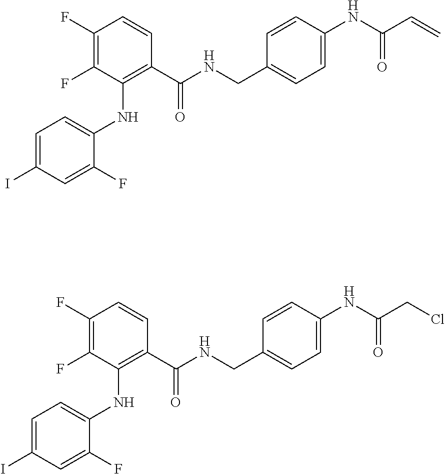 [SMILES: C=CC(=O)Nc1ccc(CNC(=O)c2ccc(F)c(F)c2Nc2ccc(I)cc2F)cc1 ; O=C(CCl)Nc1ccc(CNC(=O)c2ccc(F)c(F)c2Nc2ccc(I)cc2F)cc1]
or a pharmaceutically acceptable salt thereof.

49. A pharmaceutical composition comprising a compound of claim 48, or a pharmaceutically acceptable salt thereof, and a pharmaceutically acceptable carrier.

50. A method of inactivating ARAF in a subject in need thereof comprising administering to the subject a therapeutically effective amount of a compound of claim 48.

51. A method of treating an ARAF-mediated cancer in a subject in need thereof, comprising administering to the subject a therapeutically effective amount of a compound of claim 48, wherein the ARAF-mediated cancer may be melanoma or a carcinoma, including uterine corpus endometrial carcinoma, cholangiocarcinoma, and esophageal adenocarcinoma.

52. A compound selected from the group consisting of 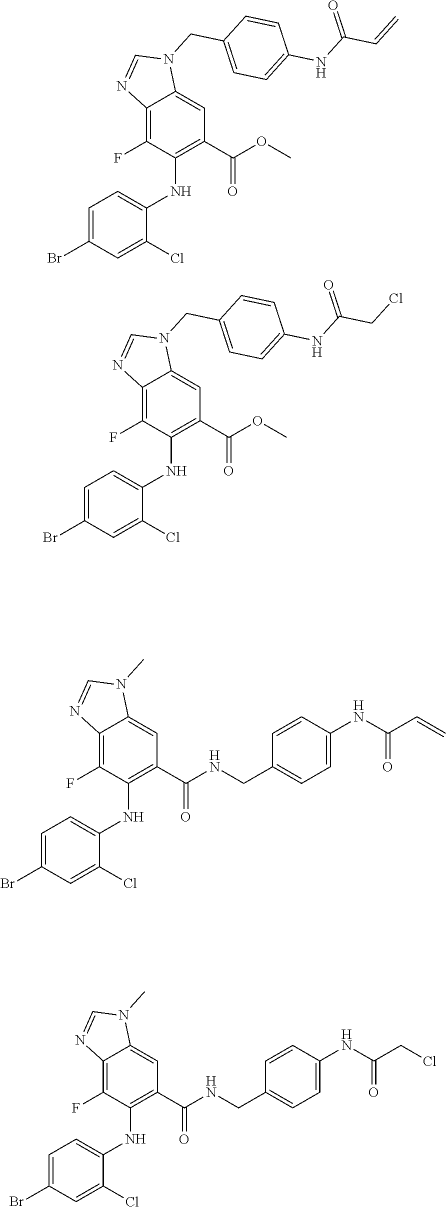 [SMILES: C=CC(=O)Nc1ccc(Cn2cnc3c(F)c(Nc4ccc(Br)cc4Cl)c(C(=O)OC)cc32)cc1 ; COC(=O)c1cc2c(ncn2Cc2ccc(NC(=O)CCl)cc2)c(F)c1Nc1ccc(Br)cc1Cl ; C=CC(=O)Nc1ccc(CNC(=O)c2cc3c(ncn3C)c(F)c2Nc2ccc(Br)cc2Cl)cc1 ; Cn1cnc2c(F)c(Nc3ccc(Br)cc3Cl)c(C(=O)NCc3ccc(NC(=O)CCl)cc3)cc21]
or a pharmaceutically acceptable salt thereof.

53. A pharmaceutical composition comprising a compound of claim 52, or a pharmaceutically acceptable salt thereof, and a pharmaceutically acceptable carrier.

54. A method of inactivating ARAF in a subject in need thereof comprising administering to the subject a therapeutically effective amount of a compound of claim 52.

55. A method of treating an ARAF-mediated cancer in a subject in need thereof, comprising administering to the subject a therapeutically effective amount of a compound of claim 52, wherein the ARAF-mediated cancer may be melanoma or a carcinoma, including uterine corpus endometrial carcinoma, cholangiocarcinoma, and esophageal adenocarcinoma.

56. A compound of Formula II: 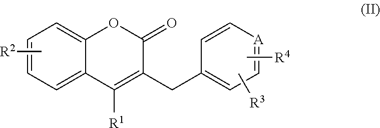
or a pharmaceutically acceptable salt thereof;
wherein
A is N, CR³, or CR⁴;
R^{t} is H or C₁₋₆ alkyl;
R² is selected from the group consisting of H, halo, 6-10 membered aryl, 5-10 membered heteroaryl, 3-10 membered cycloalkyl, 3-10 membered cycloalkenyl, 4-10 membered heterocycloalkyl, —OR⁶, —NR⁶R⁶, —SO₂R⁶, —SO₂NHR⁶, —NHSO₂R⁶, —C(O)OR⁶, —C(O)NHR⁶, —C(O)R⁶, C₁-C₆ alkyl, C₂-C₆ alkenyl, and C₂-C₆ alkynyl, wherein aryl, heteroaryl, heterocycloalkyl, cycloalkyl, alkyl, alkenyl, and alkynyl are optionally substituted one, two, or three times with R⁷;
each R³ is independently selected from the group consisting of H, halo, C₁₋₃ alkyl, C₁₋₃ haloalkyl, and C₁-3 alkoxy; and
each R⁶ is independently selected from the group consisting of H, C₁₋₆ alkyl, C₁₋₆ alkenyl, C₁₋₆ alkynyl, C₁₋₆ haloalkyl, 6-10 membered aryl, 5-6 membered heteroaryl, 3-6 membered cycloalkyl, and 3-6 membered heterocycloalkyl;
each R⁷ is independently selected from the group consisting of halo, —OH, —NH₂, —NH(C₁₋₆ alkyl), —N(C₁₋₆ alkyl)₂, and —CN;
R⁴ is selected from the group consisting of 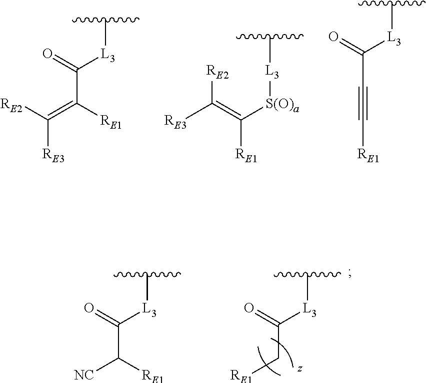
L₃ is selected from the group consisting of a bond, C₁-C₄ alkyl, and NH;
each of R_{E1}, R_{E2}, and R_{E3} is independently selected from the group consisting of H, halo, —CN, and C₁-C₆ alkyl;
a is 1 or 2; and
each z is independently 0, 1, or 2.

57. The compound of claim 56, wherein the compound of Formula II is a compound of Formula IIa: 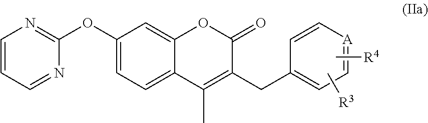
or a pharmaceutically acceptable salt thereof;
R³ is H or halo;
A is N or CH; and
R⁴

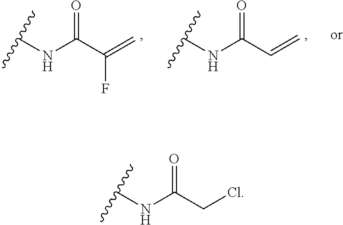

58. The compound of claim 56, wherein the compound of Formula II is selected from the group consisting of: 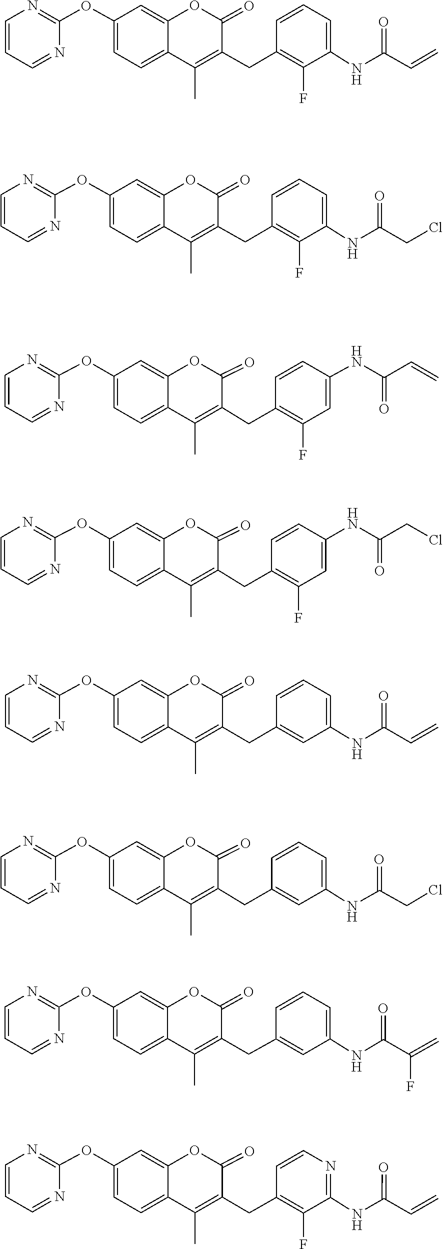 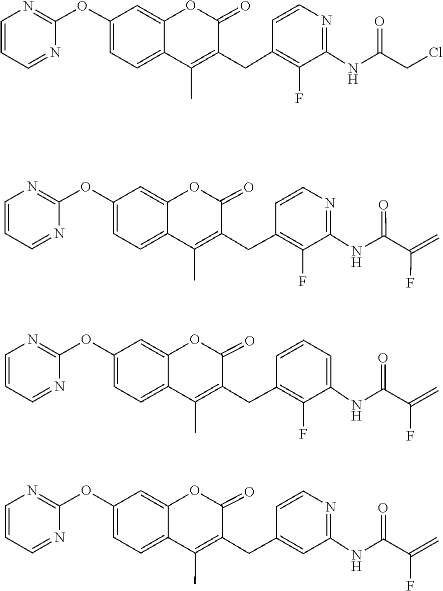 [SMILES: Cc1c(Cc2ccnc(NC(=O)CCl)c2F)c(=O)oc2cc(Oc3ncccn3)ccc12 ; C=C(F)C(=O)Nc1nccc(Cc2c(C)c3ccc(Oc4ncccn4)cc3oc2=O)c1F ; C=C(F)C(=O)Nc1cccc(Cc2c(C)c3ccc(Oc4ncccn4)cc3oc2=O)c1F ; C=C(F)C(=O)Nc1cc(Cc2c(C)c3ccc(Oc4ncccn4)cc3oc2=O)ccn1]
or a pharmaceutically acceptable salt thereof.

59. A pharmaceutical composition comprising a compound of claim 56, or a pharmaceutically acceptable salt thereof, and a pharmaceutically acceptable carrier.

60. A method of inactivating ARAF in a subject in need thereof comprising administering to the subject a therapeutically effective amount of a compound of claim 56.

61. A method of treating an ARAF-mediated cancer in a subject in need thereof, comprising administering to the subject a therapeutically effective amount of a compound of claim 56, wherein the ARAF-mediated cancer may be melanoma or a carcinoma, including uterine corpus endometrial carcinoma, cholangiocarcinoma, and esophageal adenocarcinoma.
